(19) United States
(12) Patent Application Publication
(10) Pub. No.: US 2026/0327231 A1
(43) Pub. Date: Oct. 22, 2026

(54) PROCESS FOR THE PREPARATION OF REMIMAZOLAM AND SALTS THEREOF

(71) Applicant: HARMAN FINOCHEM LIMITED, Mumbai, Maharashtra (IN)

(72) Inventors: Harpreet Singh MINHAS, Mumbai, Maharashtra (IN); Gurpreeet Singh MINHAS, Mumbai, Maharashtra (IN); Nitin Sharadchandra PRADHAN, Thane (West), Maharashtra (IN); Vijay Trimbak KADAM, Aurangabad, Maharashtra (IN); Prashant Kasturchand GODHA, Ahmednagar, Maharashtra (IN); Yogesh Pandurang ANAP, Ahmednagar, Maharashtra (IN)

(21) Appl. No.: 19/473,847

(22) PCT Filed: Apr. 1, 2024
(86) PCT No.: PCT/IN2024/050348

§ 371 Date: Oct. 9, 2025

(30) Foreign Application Priority Data
Apr. 13, 2023 (IN) 202321027324

(51) Int. Cl. C07D 487/04; C07C 269/04

(52) U.S. Cl. CPC C07D 487/04; C07C 269/04

(57) ABSTRACT

Disclosed herein is an improved process for preparation of Remimazolam and salts thereof The present invention ameliorates the use of costly reagents, cumbersome process steps employed in the art by virtue of which the present process is cost effective and industrially viable.

### FIELD OF THE INVENTION

The present invention relates to an improved process for preparation of Remimazolam and salts thereof.

### BACKGROUND OF THE INVENTION

Remimazolam (CNS7056) having IUPAC name methyl 3-{(4S)-8-bromo-1-methyl-6-(pyridin-2-yl)-4/-/-imidazo[1,2-a][1,4]benzodiazepin-4-yl}propanoate, is a benzodiazepine developed by Paion with the following chemical structure;

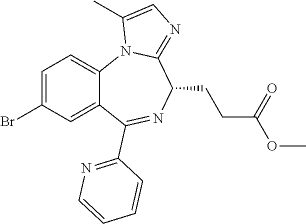 [SMILES: COC(=O)CC[C@@H]1N=C(c2ccccn2)c2cc(Br)ccc2-n2c(C)cnc21]

Remimazolam is a short-acting central nervous system depressant. It exhibits anxiolytic, amnestic, sedative, muscle relaxing, and anticonvulsant properties. Due to these properties, it is suitable for use in anesthetic practice and intensive care, such as in preoperative sedation, anxiolysis, amnestic use for perioperative cases, conscious sedation during short diagnostic, operative, or endoscopic procedures, for example, as a component for inducing and maintaining general anesthesia, before and/or along with the administration of other anesthetic agents, as well as in intensive care sedation. It is preferably administered by intravenous route.

EP Patent No. EP1183243B1 relates to benzodiazepine derivatives including Remimazolam and the examples 1c-8 disclose the preparation thereof.

U.S. Pat. No. 7,485,635B2 discloses Remimazolam and process for its preparation. The process is described in Scheme-1.

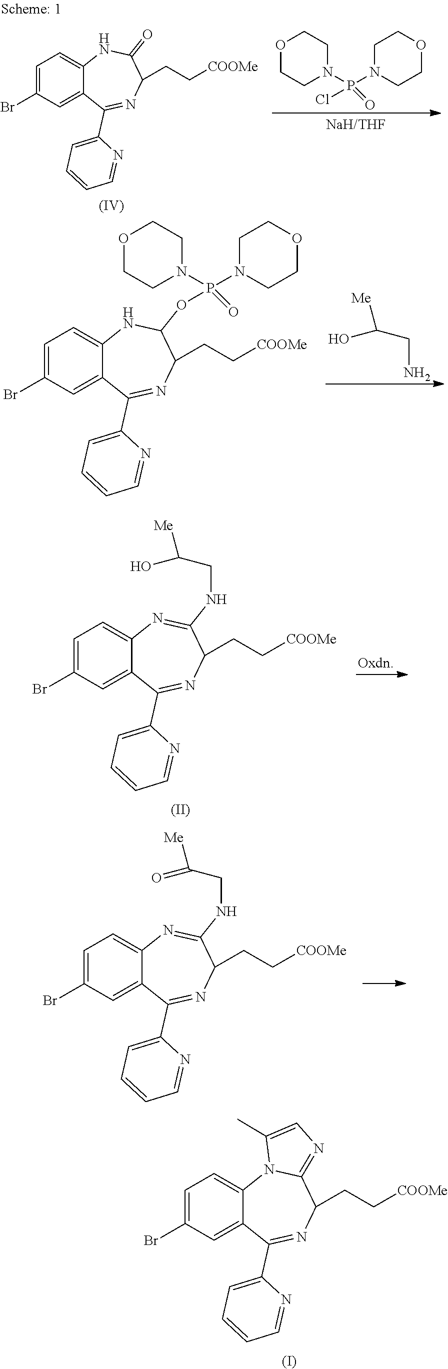

The processes described in the art are cumbersome, employ costly reagents and extensive work up procedures. Hence, a need still exists in the art to provide a process for preparation of Remimazolam and its salts which is both industrially and economically viable. This remains the objective of the invention.

### SUMMARY OF THE INVENTION

In accordance with the above, the present invention provides an improved process for preparation of Remimazolam and its salts thereof comprising;
i. Condensing 2-amino-5-bromo benzoyl pyridine (1) and (S)-5-(benzyloxy)-2-((tert-butoxycarbonyl)-amino)-5-oxopentanoic acid (2) in presence of condensing agent at a temperature ranging between −5 to 0° C. to obtain (S)-benzyl 5-((4-bromo-2-picolinoylphenyl)amino)-4-((tert-butoxycarbonyl) amino)-5-oxopentanoate(3);
ii. Cyclizing the intermediate compound (3) to yield (S)-benzyl 4-amino-5-((4-bromo-2-picolinoylphenyl)-amino)-5-oxopentanoate hydrochloride (4);
iii. Reacting the compound (4) with bismorpholino phosphinic chloride in presence of base followed by addition of 1-amino 2-propanol to yield Benzyl 3-((3S)-7-bromo-2-((2-hydroxypropyl)amino)-5-(pyridin-2-yl)-3H-benzo[e][1,4]diazepin-3-yl) propanoate (5);
iv. Oxidizing the compound (5) with suitable oxidizing agent to obtain (S)-benzyl 3-(8-bromo-1-methyl-6-(pyridin-2-yl)-4H-benzo[f]imidazo[1,2-a][1,4]diazepin-4-yl) propanoate (6);
v. Converting the compound (6) to (S)-benzyl 3-(8-bromo-1-methyl-6-(pyridin-2-yl)-4H-benzo[f]imidazo[1,2-a][1,4]diazepin-4-yl)propanoate benzenesulfonate (7);
vi. Treating compound (7) with the base to obtain (S)-benzyl 3-(8-bromo-1-methyl-6-(pyridin-2-yl)-4H-benzo[f]imidazo[1,2-a][1,4]diazepin-4-yl)propanoate (6);
vii. Transesterification of the compound (6) of step (vi) with suitable reagent to yield Remimazolam and converting to suitable acid addition salt; and viii. Purifying the crude Remimazolam acid addition salt to yield the desired product.

In an aspect, the compound (S)-5-(benzyloxy)-2-((tert-butoxycarbonyl)-amino)-5-oxopentanoic acid (2) is prepared by the process comprising;
i. Reacting benzyl alcohol with L-Glutamic acid in presence of acid to obtain (S)-2-amino-5-(benzyloxy)-5-oxopentanoic acid (i.e. L-glutamic acid benzyl ester); and
ii. Reacting (S)-2-amino-5-(benzyloxy)-5-oxopentanoic acid of step (i) with protecting agent in presence of base at a 0-5° C. to obtain (S)-5-(benzyloxy)-2-((tert-butoxycarbonyl)-amino)-5-oxopentanoic acid.

### DETAILED DESCRIPTION OF THE INVENTION

In an embodiment, the present invention relates to an improved and cost effective process for preparation of Remimazolam and its salt thereof comprising;
i. Condensing 2-amino-5-bromo benzoyl pyridine (1) and (S)-5-(benzyloxy)-2-((tert-butoxycarbonyl)-amino)-5-oxopentanoic acid (2) in presence of condensing agent at a temperature ranging between −5 to 0° C. to obtain (S)-benzyl 5-((4-bromo-2-picolinoylphenyl)amino)-4-((tert-butoxycarbonyl) amino)-5-oxopentanoate (3);

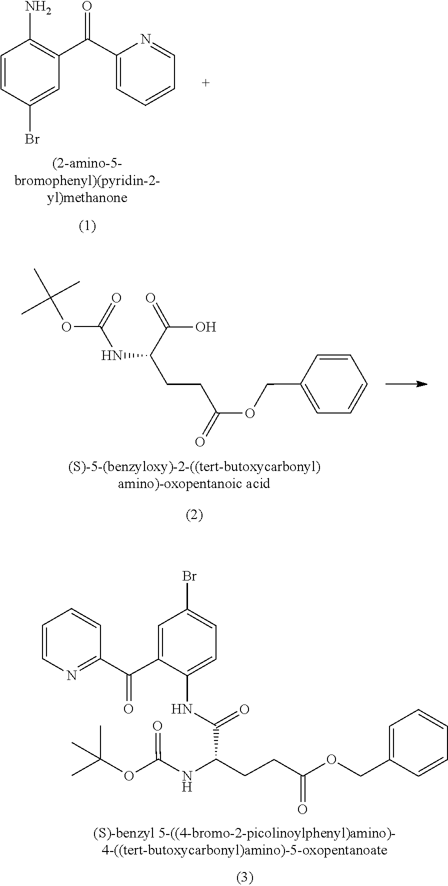 [SMILES: Nc1ccc(Br)cc1C(=O)c1ccccn1 ; CC(C)(C)OC(=O)N[C@@H](CCC(=O)OCc1ccccc1)C(=O)O ; CC(C)(C)OC(=O)N[C@@H](CCC(=O)OCc1ccccc1)C(=O)Nc1ccc(Br)cc1C(=O)c1ccccn1]

ii. Cyclizing the intermediate compound (3) to yield (S)-benzyl 4-amino-5-((4-bromo-2-picolinoylphenyl)-amino)-5-oxopentanoate hydrochloride (4);

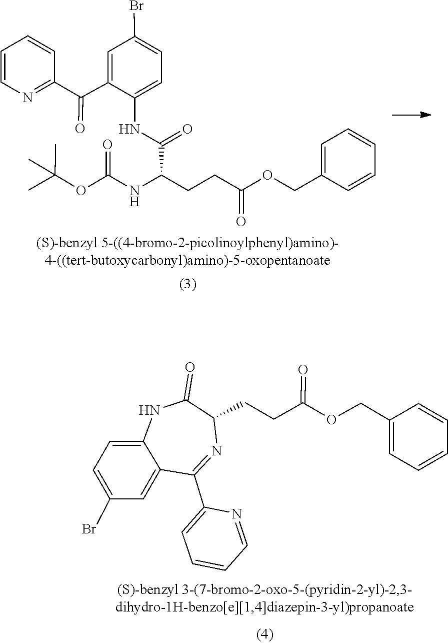 [SMILES: CC(C)(C)OC(=O)N[C@@H](CCC(=O)OCc1ccccc1)C(=O)Nc1ccc(Br)cc1C(=O)c1ccccn1 ; O=C(CC[C@@H]1N=C(c2ccccn2)c2cc(Br)ccc2NC1=O)OCc1ccccc1]

iii. Reacting the compound (4) with bismorpholino phosphinic chloride in presence of base followed by addition of 1-amino 2-propanol to yield Benzyl 3-((3S)-7-bromo-2-((2-hydroxypropyl)amino)-5-(pyridin-2-yl)-3H-benzo[e][1,4]diazepin-3-yl) propanoate (5);

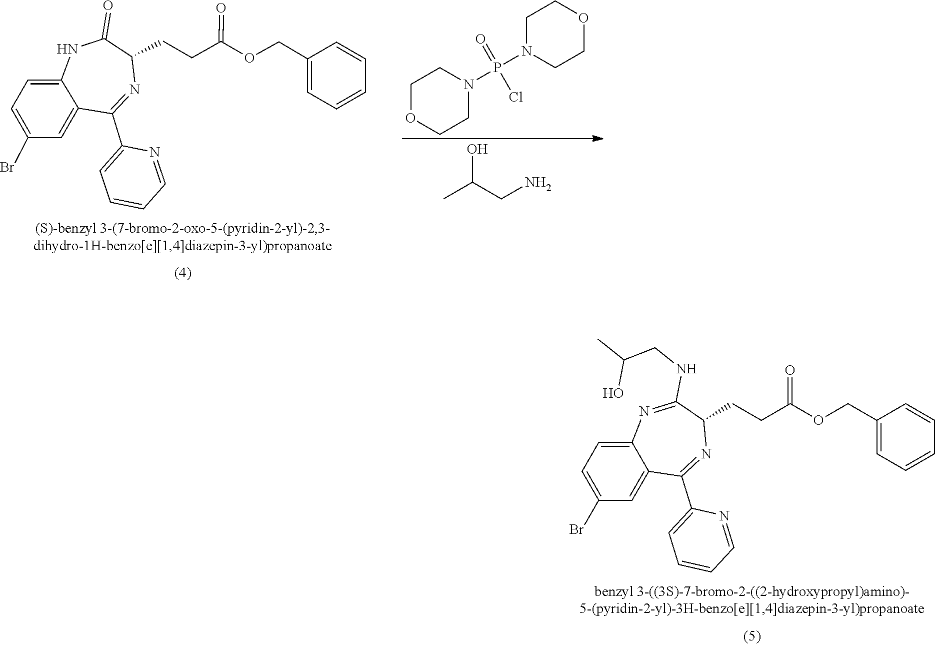

iv. Oxidizing the compound (5) with suitable oxidizing agent to obtain (S)-benzyl 3-(8-bromo-1-methyl-6-(pyridin-2-yl)-4H-benzo[f]imidazo[1,2-a][1,4]diazepin-4-yl) propanoate (6);

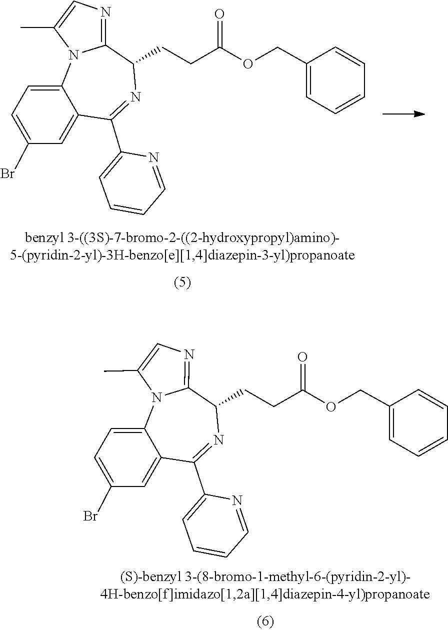 [SMILES: Cc1cnc2n1-c1ccc(Br)cc1C(c1ccccn1)=N[C@H]2CCC(=O)OCc1ccccc1 ; Cc1cnc2n1-c1ccc(Br)cc1C(c1ccccn1)=N[C@H]2CCC(=O)OCc1ccccc1]

v. Converting the compound (6) to (S)-benzyl 3-(8-bromo-1-methyl-6-(pyridin-2-yl)-4H-benzo[f]imidazo[1,2-a][1,4]diazepin-4-yl)propanoate benzenesulfonate(7);

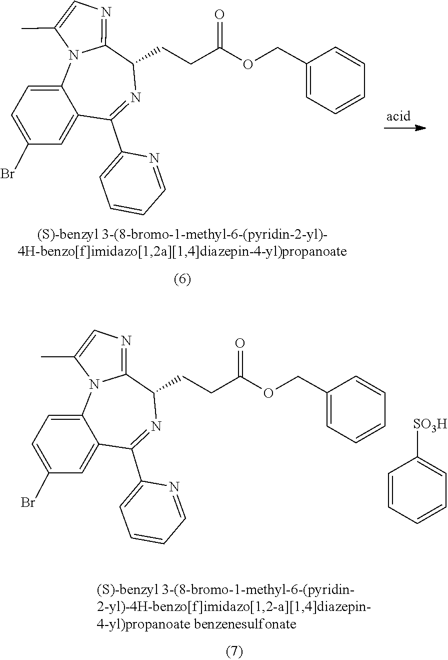

vi. Treating compound (7) with the base to obtain d (S)-benzyl 3-(8-bromo-1-methyl-6-(pyridin-2-yl)-4H-benzo[f]imidazo[1,2-a][1,4]diazepin-4-yl)propanoate (6);

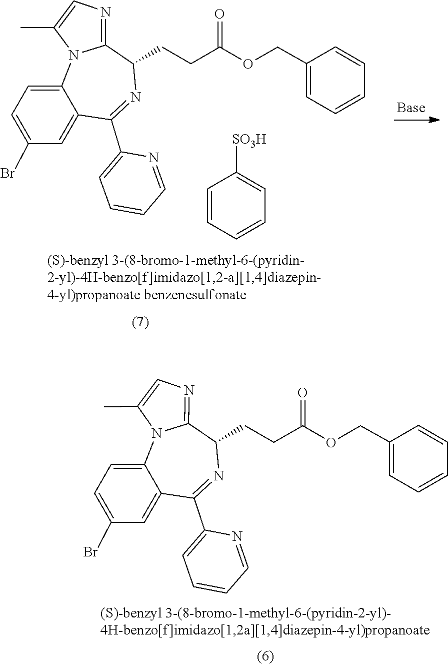

vii. Transesterification of the compound (6) with suitable reagent to yield Remimazolam and converting to suitable acid addition salt; and

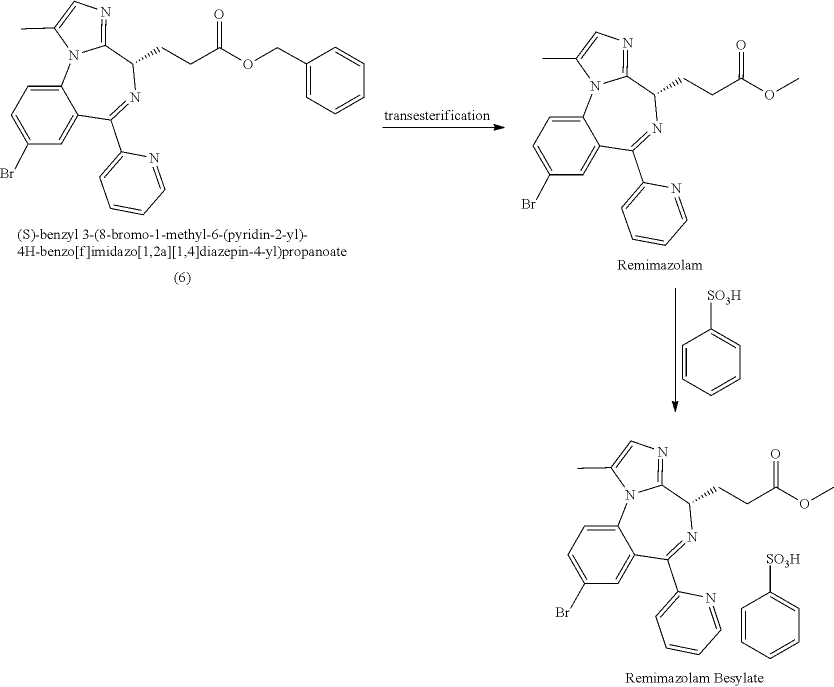

viii. Purifying the crude Remimazolam acid addition salt to yield the desired product.

According to process step (i), to the solution mixture of 2-amino-5-bromo benzoyl pyridine (1) and (S)-5-(benzyloxy)-2-((tert-butoxycarbonyl)-amino)-5-oxopentanoic acid (2) (i.e. N-Boc L-Glutamic acid-γ-benzyl ester) cooled to −5 to 0° C. is added dropwise the condensing agent selected from Dicyclohexyl carbodiimide, 1,1′-Carbonyldiimidazole (CDI) 1-ethyl-3-(3-dimethylaminopropyl) carbodiimide (EDC) and the like dissolved in the solvent at said temperature. The reaction mass is stirred for about 24 hrs at 0° C. The addition of N-Boc L-Glutamic acid-γ-benzyl ester and the condensing agent are added to the mixture intermittently at 0° C. with stirring and the reaction is monitored by HPLC. After completion of the reaction, the reaction mass was filtered and washed with suitable solvent, distilled the filtrate under vacuum to obtain (S)-benzyl 5-((4-bromo-2-picolinoylphenyl)amino)-4-((tert-butoxycarbonyl) amino)-5-oxopentanoate(3).

The process step (ii) of the present invention comprises dissolving the compound (S)-benzyl5-((4-bromo-2-picolinoylphenyl)amino)-4-((tert-butoxycarbonyl) amino)-5-oxo pentanoate (3) obtained in step (i) and cooling to a temperature of 5-10° C. followed by slowly charging 1,4 dioxane HCl to the cooled mixture and stirring for about 1-3 hours. After completion of the reaction, the reaction mass was slowly poured into the mixture of base and the solvent at 5-10° C., raised the temperature to 10-15° C. and stirred until completion of the reaction. The reaction mass was then filtered, washed, distilled out the solvent under vacuum and the crude compound was further purified to obtain (S)-benzyl 4-amino-5-((4-bromo-2-picolinoylphenyl)-amino)-5-oxopentanoate hydrochloride (4).

According to the process step (iii), to the solution of the compound (4) and dry THE was added a base selected from Lithium Diisopropylamide (LDA) or sodium hydride at low temperature of −20 to −15° C. The reaction mixture was stirred for about 1.5 to 3 hours at 0° C. followed by addition of bismorpholino phosphinic chloride (BMPC) at said temperature and stirring the mixture for another 2 to 3 hours. 1-amino 2-propanol was charged to the reaction mass at 0° C. and stirring the reaction overnight at room temperature. Distilling out the solvent under vacuum and followed by addition of suitable solvent and washing. Distilling out the solvent from the organic layer under vacuum to obtain the crude compound which was further purified to yield Benzyl 3-((3S)-7-bromo-2-((2-hydroxypropyl)amino)-5-(pyridin-2-yl)-3H-benzo [e][1,4]diazepin-3-yl) propanoate (5).

The process step (iv) of the present invention comprises adding to the solution of compound (5) and THE cooled to 15-25° C., suitable mild oxidizing agent selected from Dess Martin Periodinane or Pyridinium dichromate and stirring the reaction mass for about 2-3 hours. After completion of the reaction, reaction mass was filtered and washed. Collected the filtrate and distilled out THE completely, washed with the solvent, distilling out the solvent to obtain (S)-benzyl 3-(8-bromo-1-methyl-6-(pyridin-2-yl)-4H-benzo[f]imidazo[1,2-a][1,4]diazepin-4-yl) propanoate (6).

The process step (v) comprises dissolving the compound (6) in a suitable solvent followed by addition of organic acid selected from methane sulphonic acid, ethane sulphonic acid and the like in said solvent. Stirring the reaction mass for about 2 hours at room temperature, filtering, washing with suitable solvent and drying to obtain the suitable acid salt of compound (7).

According to step (vi), to the suitable acid salt compound (7) was charged a mixture of water and organic solvent. The pH was adjusted to 10-12 with a base selected from alkali or alkaline earth metal carbonates or bicarbonates. The layers were separated, washed the organic layer, distilled out the solvent under vacuum to yield the compound (6) as free base.

The step (vii) comprises preparing (S)-methyl 3-(8-bromo-1-methyl-6-(pyridin-2-yl)-4H-benzo[f]imidazo[1,2-a][1,4]diazepin-4-yl)propanoate (Remimazolam) by trans esterification. Accordingly the compound (6) was charged in a solvent and cooled to 5-10° C. Further, 15-20% methanolic HCl was charged drop wise in reaction mass at 5-10° C. and the reaction mass was stirred at room temperature. After completion of reaction, reaction mass was cooled to 0-5° C. and water was charged slowly to the mixture at same temperature. The pH was adjusted to 7.0 to 8.0 with alkali or alkaline earth metal carbonates or bicarbonates. Distilled out methanol from the reaction mass under vacuum at 35-40° C. Suitable solvent was charged to the mixture and stirred, separated the layers and further washed. Distilled out the solvent under vacuum to yield the desired product.

In an embodiment, Remimazolam obtained by the process of the present invention was further converted to salts such as mesylate, besylate using suitable acids.

In yet another embodiment, the Remimazolam salts were purified by charging said Remimazolam salt in an organic solvent and boiling to reflux temperature. Cooled the mixture to room temperature and stirred. Filtered the product and washed with the solvent, dried under vacuum to obtain pure Remimazolam salt.

In another embodiment, the solvent for the process of the present invention is selected from polar or non-polar, protic or aprotic solvents which include but is not limited to alcohols selected from lower aliphatic C1 to C5 alcohols, aromatic alcohols such as benzyl alcohol, ethers such as DMF, THF, dimethyl ether, 1,4 dioxane and the like; hydrocarbons selected from aliphatic or aromatic hydrocarbons halogenated hydrocarbons such as methylene dichloride (MDC), ethylene dichloride, chloroform, carbon tetrachloride and the like; ketones, esters, nitriles and the like alone or combination thereof.

In an embodiment, the present process is characterized by presence of benzyl ester Impurity-A, benzyl 3-[(4S)-8-bromo-1-methyl-6-(pyridin-2-yl)-4H-imidazo[1,2-a][1,4]benzodiazepin-4-yl]propanoate; acid impurity-B3-[(4S)-8-bromo-1-methyl-6-(pyridin-2-yl)-4H-imidazo[1,2-a][1,4]benzodiazepin-4-yl]propanoic acid and ethyl ester impurity-C, ethyl 3-[(4S)-8-bromo-1-methyl-6-(pyridin-2-yl)-4H-imidazo[1,2-a][1,4]benzodiazepin-4-yl]propanoate.

In yet another embodiment, the present invention relates to preparation of (S)-5-(benzyloxy)-2-((tert-butoxycarbonyl)-amino)-5-oxopentanoic acid (2) comprising;
i. Reacting benzyl alcohol with L-Glutamic acid in presence of acid to obtain (S)-2-amino-5-(benzyloxy)-5-oxopentanoic acid (i.e. L-glutamic acid benzyl ester); and
ii. Reacting (S)-2-amino-5-(benzyloxy)-5-oxopentanoic acid of step (i) with protecting agent in presence of base at a 0-5° C. to obtain (S)-5-(benzyloxy)-2-((tert-butoxycarbonyl)-amino)-5-oxopentanoic acid.

Accordingly, to the mixture of benzyl alcohol and L-Glutamic acid was added drop wise acid selected from methane sulphonic acid, ethane sulphonic acid or benzyl sulphonic acid. at a temperature ranging between 40-50° C. Maintained the reaction mass for about 2 hours at said temperature followed by cooling to 25-30° C. Adding water to the reaction mass and separating the layers. Adding suitable solvent to the aqueous layer and adjusting the pH with suitable base. Filtering the solid, washing and drying under vacuum to obtain (S)-2-amino-5-(benzyloxy)-5-oxopentanoic acid (i.e. L-glutamic acid benzyl ester).

L-Glutamic acid-γ-benzyl ester so obtained was charged in mixture of water and solvent and cooled to 0-5° C. Suitable protecting agents are selected from boc-anhydride, Fmoc chloride, Phthalic anhydride or tert butoxycarbonyl and such like in presence of the base selected from organic base such as ethylamine, triethylamine, pyridine and the like; preferably triethylamine were added to mix of L-Glutamic acid-γ-benzyl ester at said temperature. The reaction mass was stirred overnight at room temperature, distilled the solvent, washed and dried to obtain (S)-5-(benzyloxy)-2-((tert-butoxycarbonyl)-amino)-5-oxopentanoic acid (2).

The solvent for the process of preparation of the compound (S)-5-(benzyloxy)-2-((tert-butoxycarbonyl)-amino)-5-oxopentanoic acid (2) in the present invention was selected from water, aliphatic or aromatic hydrocarbons, ethers and the like alone or combination thereof.

The invention will now be described in the following specific examples, however, it is being understood that the particulars shown are solely for purpose of illustrative discussion of preferred embodiments of the invention.

### EXAMPLES

### Example-1: Preparation of (S)-2-amino-5-(benzyloxy)-5-oxopentanoic acid (i.e. L-Glutamic Acid Benzyl Ester)

Benzyl alcohol (55 g) was added in L-Glutamic acid (50 g suspended in toluene). Methane Sulphonic acid (39.2 g) was added drop wise in the reaction mass at 45° C. Maintained the reaction mass for 2 hrs and cooled to 25-30° C. and stirred for 3 hrs. Charged water (100) ml in the reaction mass and separated the layers. Ethanol (50 ml) was added in the aqueous layer and pH was adjusted to 6.0 using liquor ammonia. Filtered the solid and washed with Ethanol:water mixture (1:1). Solid was dried under vacuum at 45° C. to obtain the title product (44 g).

### Example-2: Preparation of (S)-5-(benzyloxy)-2-((tert-butoxycarbonyl)-amino)-5-oxopentanoic acid

L-Glutamic acid-γ-benzyl ester (44 g) charged in mixture of water (440 ml) and DMF (440 ml). Cooled the reaction mass to 0-5° C. and charged Boc anhydride (41 g) and triethylamine (19.2 g) in the reaction mass at 0-5° C. Slowly raised the temperature of the reaction mass to room temperature. Stirred the reaction mass overnight at room temperature. Distilled out the solvent from the reaction mass under vacuum. Charged 300 ml ethyl acetate and 100 ml water in degas mass. Stirred and cooled the reaction mass to 0-5° C. Adjusted pH=1.5-2.5 using 1N HCl. Stirred for 10 min and separated the layers. Aqueous layer re-extracted with 250 ml ethyl acetate. Combined both organic layer and washed with water and sodium chloride solution. Distilled out the solvent under vacuum. Stirred the residue in heptane. Filtered the product and dried in vacuum oven at 40° C. Yield=50 g

### Example-3: Preparation of (S)-benzyl 5-((4-bromo-2-picolinoylphenyl)amino)-4-((tert-butoxycarbonyl) amino)-5-oxopentanoate

To a clean and dried RBF, charged methylene chloride (800 ml), 2-amino-5-bromo benzoyl pyridine (100 g) and N-Boc L-Glutamic acid-γ-benzyl ester (146 g) obtained from Example-2), cooled the reaction mass to −5 to 0° C. Dicyclohexyl carbodiimide (89.3 g) dissolved in 100 ml methylene dichloride added drop wise in reaction mass at −5 to 0° C. Stirred the reaction mass for 24 hrs at 0° C. Charged 60 g N-Boc L-Glutamic acid 7-benzyl ester and 37.2 g Dicyclohexyl carbodiimide in reaction mass at 0° C. Stirred the reaction mass for 24 hrs. Charged 30.4 g N-Boc L-Glutamic acid γ-benzyl ester and 18.6 g Dicyclohexyl carbodiimide in reaction mass at 0° C. Stirred the reaction mass for another 24 hrs. Monitored the reaction by HPLC. Filtered the reaction mass and washed the solid with methylene dichloride. Distilled out the filtrate ML under vacuum. Yield=300 g

### Example-4: (S)-benzyl 4-amino-5-((4-bromo-2-picolinoylphenyl)-amino)-5-oxopentanoate hydrochloride

The compound obtained from Example-3 (300 g) was dissolved in methylene dichloride (450 ml). Cooled to 5-10° C. and slowly charged 1,4-Dioxane HCl (470 ml). Stirred the reaction mass for 1-3 hrs. After completion of reaction, reaction mass was slowly poured in acetonitrile (1000 ml) acetonitrile with sodium bicarbonate (600 g) at 5-10° C. Reaction mass was maintained under stirring for 3 hrs at 10-15° C. After completion of the reaction, filtered the reaction mass and washed the solid with acetonitrile. Distilled out the solvent under vacuum at 35-40° C. The crude material was purified by column to obtain the title compound (150 g).

### Example-5: Benzyl 3-((3S)-7-bromo-2-((2-hydroxypropyl)amino)-5-(pyridin-2-yl)-3H-benzo[e][1,4]diazepin-3-yl)propanoate

The solution of the compound obtained from Example-4 (250 g) and dry THE (1000 ml) was charged in Lithium diisopropylamide (LDA) (1.05 mol) at −20 to −15° C. Stirred the reaction for 1.5 to 2 hrs at 0° C. Charged bismorpholino phosphoryl chloride (BMPC-306 g) to the reaction mass at 0° C. and stir for 2-3 hrs. 1-amino 2-propanol (80 g) was charged in reaction mass at 0° C. Stirred reaction for overnight at room temperature. Second lot of 1-amino 2-propanol (27.5 g) was charged in reaction mass at room temperature. Stirred the reaction for 24 hrs at room temperature. Distilled out THE under vacuum at 35-40° C. Charged MDC (1250 ml) and washed with saturated solution of sodium bicarbonate (1000 ml). Separated the layers and washed MDC layer with 500 ml saturated solution of ammonium chloride. Distilled out MDC under vacuum at 35-40° C. to obtain the title compound (335 g).

### Example-6: (S)-benzyl 3-(8-bromo-1-methyl-6-(pyridin-2-yl)-4H-benzo[f]imidazo[1,2-a][1,4]diazepin-4-yl)propanoate

The solution of the compound obtained from Example-5 (330 g) and THE (2.5 L) was cooled to 15-25° C. Charged Dess Martin Periodinane (275 g) and stirred the reaction mass for 1 hr. After completion of the reaction, reaction mass was filtered and washed with THF. Collected filtrate and distilled out THE completely. Charged ethyl acetate (3.0 L) and washed with saturated solution of sodium bicarbonate (660 ml). Stirred, settled and separated the layers. Washed ethyl acetate with saturated solution of ammonium chloride. The product is extracted in 1N HCl solution. Ethyl acetate is charged to the HCl solution and product is extracted in Ethyl acetate at basic pH by sodium hydroxide. Layers are separated and distilled out ethyl acetate under vacuum at 30-40° C. to obtain the title compound (180 g).

### Example-6a: Oxidation Using Pyridinium Dichromate

The compound (1.0 g) obtained from Example 5 was dissolved in MDC (20 ml). Charged 2.5 g pyridinium dichromate (2.5 g) and stirred reaction mass for 24 hrs. After completion of the reaction, charged MTBE (40 ml). Stirred reaction mass for 30 min. at room temperature. Filtered the solid and washed filtrate twice with water (10 ml). Separated the layers and distilled out MTBE under vacuum below 40° C. to obtain the title compound (0.8 g).

### Example-7: (S)-benzyl 3-(8-bromo-1-methyl-6-(pyridin-2-yl)-4H-benzo[f]imidazo[1,2-a][1,4]diazepin-4-yl)propanoate benzenesulfonate

The compound (175 g) from Example-6 was dissolved ethyl acetate (1000 ml). Charged slowly solution of benzene sulphonic acid (53.7 g dissolved in 150 ml ethyl acetate). Stirred the reaction mass for 2 hrs at room temperature. Filtered and washed the product with ethyl acetate (175 ml). Dried the product at 40-45° C. to obtain the title compound (180 g).

### Example-8: (S)-benzyl 3-(8-bromo-1-methyl-6-(pyridin-2-yl)-4H-benzo[f]imidazo[1,2-a][1,4]diazepin-4-yl)propanoate

The compound (180 g) obtained from Example-8 is charged to ethyl acetate (900 ml) and water (900 ml). pH was adjusted to 10-12 with aqueous solution of potassium carbonate. Layers are separated and washed the organic layer with water (360 ml) and 20% sodium chloride solution (360 ml). Distilled out organic layer under vacuum to yield the title compound (135 g).

### Example-9: Preparation of (S)-methyl 3-(8-bromo-1-methyl-6-(pyridin-2-yl)-4H-benzo[f]imidazo[1,2-a][1,4]diazepin-4-yl)propanoate (Remimazolam)

The compound obtained from Example-6 (13 g) was charged in methanol (130 ml). Cooled the reaction mass to 5-10° C. 15-20% Methanolic HCl (13 ml) was charged drop wise in reaction mass at 5-10° C. Reaction mass was stirred for 24 hrs at room temperature. After completion of the reaction, distilled out Methanol under vacuum at 35-40° C. Charged Methylene chloride (130 ml) in residue and washed with saturated solution of sodium bicarbonate (130 ml). Aqueous layer was extracted with Methylene Dichloride (65 ml). Organic Layers were combined and washed with water followed by 20% sodium chloride solution. Distilled out methylene Chloride under vacuum at 35-40° C. Yield=6.5 g

### Example-10: Preparation of Remimazolam Besylate (Crude)

Remimazolam (100 g) was dissolved in ethyl acetate (400 ml) and cooled to 15-20° C. Charged solution of benzene Sulphonic acid (33.1 g) in ethanol (250 ml). Stirred the reaction for 1-2 hrs at room temperature. Filtered and washed the product with ethyl acetate. Dried the product under vacuum at 40-45° C. to obtain Remimazolam Besylate (110 g).

HPLC purity=99.23%,

### Example-11: Purification of Crude Remimazolam Besylate

Charged Remimazolam Besylate (100 g) in aqueous acetone (1000 ml). Raised the temperature of reaction mass to reflux and maintained for 30 min. Cooled to room temperature and stirred for 1 hr. Filtered the product and washed with acetone (100 ml). Dried the product under vacuum at 40-45° C. to obtain pure Remimazolam Besylate (90 g).

HPLC purity=99.72%,

Remimazolam Besylate impurity profile

| | | | | Impurity |
|---|---|---|---|---|
| | | | | profile as |
| Sr. | Impurity | | | per present |
| No. | name | IUPAC Name | Structure | invention |
| 1. | Impurity- A Benzyl ester impurity | benzyl 3-[(4S)-8- bromo-1-methyl-6- (pyridin-2-yl)-4H- imidazo[1,2- a][1,4]benzodiazepin- 4-yl]propanoate Mol. wt. 515.40 | 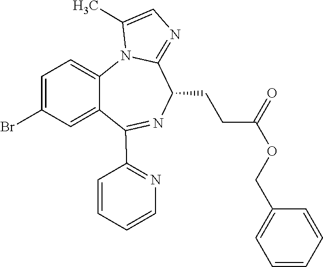 [SMILES: Cc1cnc2n1-c1ccc(Br)cc1C(c1ccccn1)=N[C@H]2CCC(=O)OCc1ccccc1] | 0.04% |
| 2. | Impurity- B Acid impurity | 3-[(4S)-8-bromo-1- methyl-6-(pyridin-2- yl)-4H-imidazo[1,2- a][1,4]benzodiazepin- 4-yl]propanoic acid Mol. wt. 425.28 | 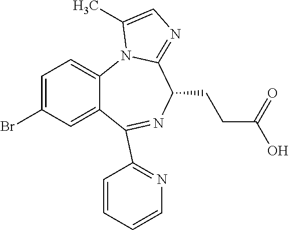 [SMILES: Cc1cnc2n1-c1ccc(Br)cc1C(c1ccccn1)=N[C@H]2CCC(=O)O] | 0.07% |
| 3. | Impurity- C Ethyl ester impurity | ethyl 3-[(4S)-8-bromo- 1-methyl-6-(pyridin-2- yl)-4H-imidazo[1,2- a][1,4]benzodiazepin- 4-yl]propanoate Mol. wt. 453.33 | 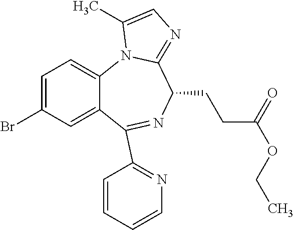 [SMILES: CCOC(=O)CC[C@@H]1N=C(c2ccccn2)c2cc(Br)ccc2-n2c(C)cnc21] | 0.06% |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. An improved process for the preparation of remimazolam or a salt thereof comprising;
i. condensing 2-amino-5-bromo benzoyl pyridine (1) and (S)-5-(benzyloxy)-2-((tert-butoxycarbonyl)-amino)-5-oxopentanoic acid (2) in the presence of a condensing agent at a temperature ranging between −5° C. to 0° C. to obtain an intermediate compound (3) (S)-benzyl 5-((4-bromo-2-picolinoylphenyl)amino)-4-((tert-butoxycarbonyl) amino)-5-oxopentanoate; 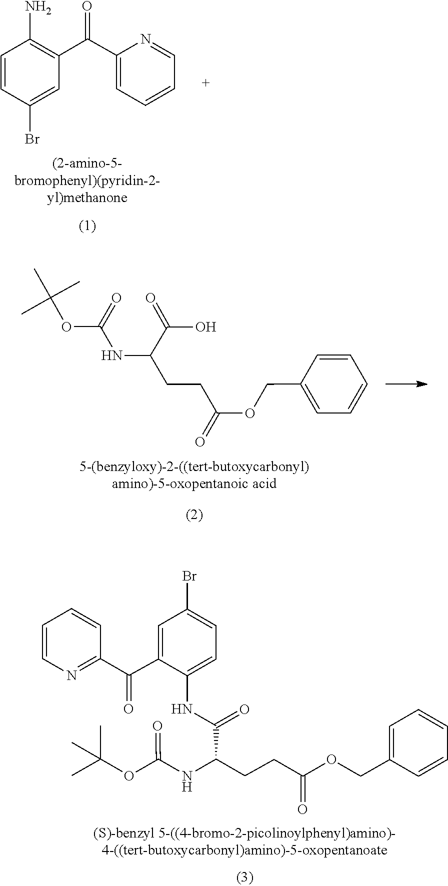 [SMILES: Nc1ccc(Br)cc1C(=O)c1ccccn1 ; CC(C)(C)OC(=O)NC(CCC(=O)OCc1ccccc1)C(=O)O ; CC(C)(C)OC(=O)N[C@@H](CCC(=O)OCc1ccccc1)C(=O)Nc1ccc(Br)cc1C(=O)c1ccccn1]
ii. cyclizing the intermediate compound (3) to yield a compound (4) (S)-benzyl 4-amino-5-((4-bromo-2-picolinoylphenyl)-amino)-5-oxopentanoate hydrochloride; 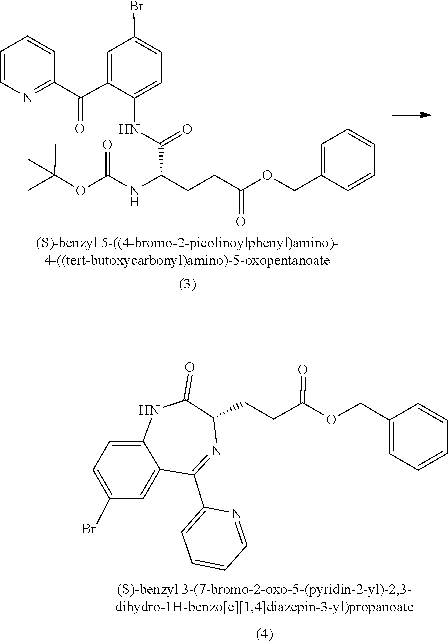 [SMILES: CC(C)(C)OC(=O)N[C@@H](CCC(=O)OCc1ccccc1)C(=O)Nc1ccc(Br)cc1C(=O)c1ccccn1 ; O=C(CC[C@@H]1N=C(c2ccccn2)c2cc(Br)ccc2NC1=O)OCc1ccccc1]
iii. reacting the compound (4) with bismorpholino phosphinic chloride in the presence of abase followed by addition of 1-amino 2-propanol to yield a compound (S) benzyl 3-((3S)-7-bromo-2-((2-hydroxypropyl)amino)-5-(pyridin-2-yl)-3H-benzo[e][1,4]diazepin-3-yl) propanoate; 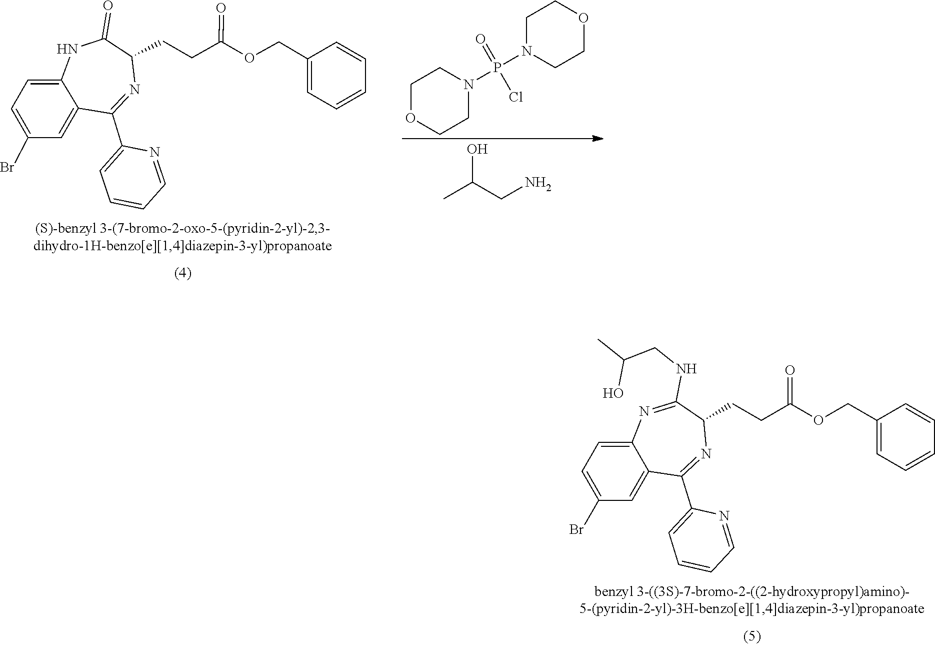
iv. oxidizing the compound (5) with an oxidizing agent to obtain a compound (6) (S)-benzyl 3-(8-bromo-1-methyl-6-(pyridin-2-yl)-4H-benzo[f]imidazo[1,2-a][1,4]diazepin-4-yl)propanoate; 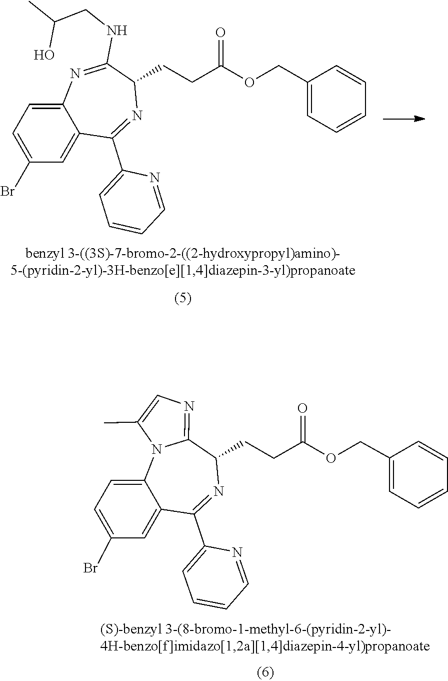 [SMILES: CC(O)CNC1=Nc2ccc(Br)cc2C(c2ccccn2)=N[C@H]1CCC(=O)OCc1ccccc1 ; Cc1cnc2n1-c1ccc(Br)cc1C(c1ccccn1)=N[C@H]2CCC(=O)OCc1ccccc1]
v. converting the compound (6) with a reagent to a compound (7) (S)-benzyl 3-(8-bromo-1-methyl-6-(pyridin-2-yl)-4H-benzo[f]imidazo[1,2-a][1,4]diazepin-4-yl) propanoate benzenesulfonate; 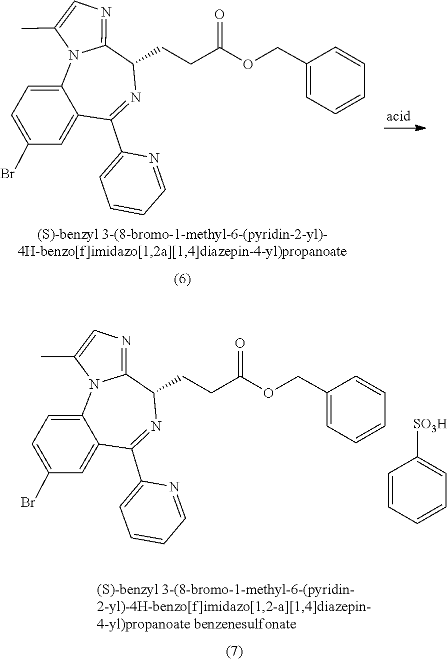
vi. treating compound (7) with a base to obtain the compound (6) (S)-benzyl 3-(8-bromo-1-methyl-6-(pyridin-2-yl)-4H-benzo[f]imidazo[1,2-a][1,4]diazepin-4-yl) propanoate; 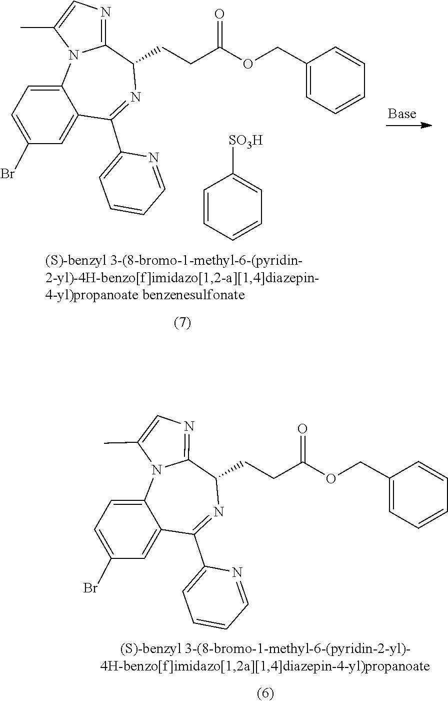
vii. transesterification of the compound (6) with a reagent to yield remimazolam and converting the remimazolam to a crude remimazolam acid addition salt; and 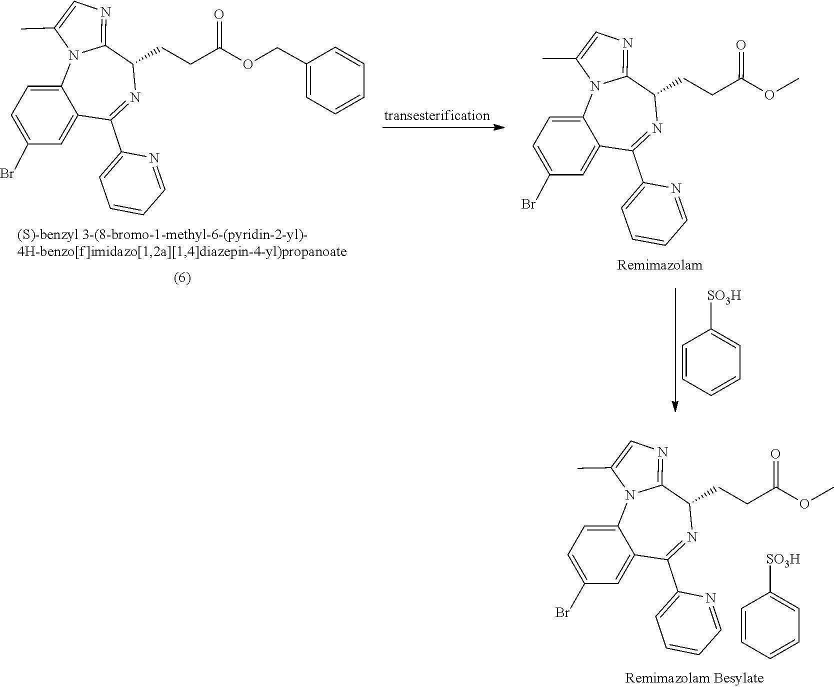
viii. purifying the crude remimazolam acid addition salt.

2. The process as claimed in claim 1, wherein the condensing agent in step (i) comprises dicyclohexyl carbodiimide, 1,1′-carbonyldiimidazole, or 1-ethyl-3-(3-dimethylaminopropyl) carbodiimide.

3. The process as claimed in claim 1, wherein the oxidizing agent in step (iv) comprises dess martin periodinane or pyridinium dichromate.

4. The process as claimed in claim 1, wherein a solvent for the process comprises water, an alcohol; an ether; a hydrocarbon; a halogenated hydrocarbon; a ketones; an ester; a nitrile; or a combination thereof.

5. The process as claimed in claim 1, wherein the base is comprises an alkali metal carbonate, an alkali metal bicarbonate, liquor ammonia, lithium diisopropylamide, sodium hydride, or a combination thereof.

6. A process for the preparation of (S)-5-(benzyloxy)-2-((tert-butoxycarbonyl)-amino)-5-oxopentanoic acid (2) comprising;
i. reacting benzyl alcohol with L-Glutamic acid in the presence of an acid to obtain (S)-2-amino-5-(benzyloxy)-5-oxopentanoic acid (i.e. L-glutamic acid benzyl ester); and
ii. reacting(S)-2-amino-5-(benzyloxy)-5-oxopentanoic acid of step (i) with a protecting agent in the presence of base at a 0° C.-5° C. to obtain (S)-5-(benzyloxy)-2-((tert-butoxycarbonyl)-amino)-5-oxopentanoic acid.

7. The process as claimed in claim 6, wherein the acid in step (i) comprises methane sulphonic acid, ethane sulphonic acid, or benzyl sulphonic acid.

8. The process as claimed in claim 6, wherein the protecting agent in step (ii) comprises boc-anhydride, Fmoc chloride, phthalic anhydride, or tert butoxycarbonyl.

9. The process as claimed in claim 6, wherein the base in step (ii) comprises an organic base.

10. The process as claimed in claim 6, wherein the solvent for the process comprises water, an aliphatic hydrocarbon, an aromatic hydrocarbon, an ether, or a combination thereof.

11. The process as claimed in claim 1, wherein said process is characterized by the impurities;
i. benzyl 3-[(4S)-8-bromo-1-methyl-6-(pyridin-2-yl)-4H-imidazo[1,2-a][1,4]benzodiazepin-4-yl]propanoate as Impurity A in an amount of about 0.04%;
ii. 3-[(4S)-8-bromo-1-methyl-6-(pyridin-2-yl)-4H-imidazo[1,2-a][1,4]benzodiazepin-4-yl]propanoic acid as Impurity B in an amount of about 0.07%; and
iii. ethyl 3-[(4S)-8-bromo-1-methyl-6-(pyridin-2-yl)-4H-imidazo[1,2-a][1,4]benzodiazepin-4-yl]propanoate in an amount of about 0.06%.

12. The process as claimed in claim 4, wherein the solvent comprises an alcohol comprising a lower aliphatic C1 to C5 alcohol, an aromatic alcohol, or a combination thereof.

13. The process as claimed in claim 12, wherein the aromatic alcohol comprises benzyl alcohol.

14. The process as claimed in claim 4, wherein the solvent comprises an ether comprises DMF, THF, methyl tert-butyl ether, 1,4 dioxane, or a combination thereof.

15. The process as claimed in claim 4, wherein the solvent comprises a hydrocarbon comprising an aliphatic hydrocarbon, an aromatic hydrocarbon, or a combination thereof.

16. The process as claimed in claim 4, wherein the solvent comprises a hydrocarbon comprising a halogenated hydrocarbon comprising methylene dichloride, ethylene dichloride, chloroform, carbon tetrachloride, or a combination thereof.

17. The process as claimed in claim 9, wherein the organic base comprises ethylamine, triethylamine, or pyridine.
